(19) United States
(12) Patent Application Publication
(10) Pub. No.: US 2024/0317666 A1
(43) Pub. Date: Sep. 26, 2024

(54) METHOD FOR THE RECYCLING OF A TEXTILE WASTE COMPRISING A CELLULOSIC COMPONENT AND A POLYESTER COMPONENT

(71) Applicant: GR3N SA, Lugano (CH)

(72) Inventor: Maurizio Crippa, Albese con Cassano (CO) (IT)

(21) Appl. No.: 18/276,791

(22) PCT Filed: Mar. 14, 2022
(86) PCT No.: PCT/IB2022/052253

§ 371 Date: Aug. 10, 2023

(30) Foreign Application Priority Data
Mar. 15, 2021 (IT) 102021000006077

(51) Int. Cl. C07C 51/41; C07C 63/28; C08J 11/14; C08J 11/18

(52) U.S. Cl. CPC C07C 51/412; C07C 63/28; C08J 11/14; C08J 11/18; C08J 2301/02

(57) ABSTRACT

The present invention relates to a method for the recycling of a textile waste (ST) comprising a cellulosic component and a polyester component, comprising the following steps: —a depolymerization step (110), comprising submitting the textile waste (ST) to a depolymerization reaction of the polyester component by basic hydrolysis by means of a solvolytic mixture (MS) comprising an alkali metal hydroxide (IMA) and at least one water-soluble organic reaction solvent (SOIR) until obtaining a biphasic mixture (MB) comprising an initial solid phase (ESI), containing a terephthalic acid salt (SAT) and cellulosic fibres, and an initial liquid phase (ELI), containing the water-soluble organic reaction solvent (SOIR): —a first step of solid-liquid separation (210), comprising the removal of at least a part of the initial liquid phase (ELI) from the biphasic mixture (MB); —a step of treatment with organic solvent (220), wherein said initial solid phase (ESI), following the first step of solid-liquid separation (210), is treated with an organic washing solvent (SOL) until removing, from the initial solid phase (ESI), one or more possible contaminants present therein and obtaining a solid purified phase (ESP) containing the terephthalic acid salt (SAT) and the cellulosic fibres; —a second step of solid-liquid separation (230), wherein the solid purified phase (ESP) is separated from the organic washing solvent (SOL) and from the one or more possible contaminants dissolved therein: —a step of treatment with water (240)), comprising bringing the solid purified phase (ESP) into contact with washing water (AL) until solubilizing the terephthalic acid salt (SAT) contained therein and obtaining, by the removal of the later, a final solid phase (FSF) comprising said cellulosic fibres.

The present invention relates to a method for the recycling of a textile waste comprising a cellulosic component (preferably cotton) and a polyester component.

Nowadays, it is known in the textile field to use mixed fabrics that comprise both natural and synthetic fibres, joined together.

An example of such fabrics is the so-called “Polycotton”, a fabric comprising cotton fibres and polyester fibres in a variable ratio, and possible other substances such as, for example, dyes.

In addition to being the most common type of polyester fabric on the market, Polycotton represents a very important part of the entire textile field currently available on the world market.

The large quantity of Polycotton produced every year poses the problem of how to recycle it, both in relation to the cotton component and the polyester component; this problem is increasingly felt, in particular from the point of view of the so-called “circular economy”.

In order to recycle Polycotton, processes of mechanical separation of the cotton fibres from the polyester fibres are known; however, these known solutions are not satisfactory as they do not allow polyester monomers to be obtained, nor possible contaminants which limit the possibilities of reuse, such as dyes, to be removed from the cotton.

The main task of the present invention is to obtain a method for the recycling of a textile waste comprising a cellulosic component (preferably cotton) and a polyester component, which allows effectively separating the cellulosic component from the polyester component and from possible contaminants present in said textile waste.

A further purpose of the present invention is that it can be applied to the industrial-scale recovery of the cellulosic and/or polyester component contained in such textile waste.

Another purpose of the invention is to obtain a process for the recycling of a textile waste comprising a cellulosic component (preferably cotton) and a polyester component that is relatively simple, effective and economical.

Another purpose again is to obtain a process for the recycling of a textile waste comprising a cellulosic component (preferably cotton) and a polyester component, which is relatively sustainable and which makes effective use of possible ancillary chemical substances in the process.

These and other purposes according to the present invention are achieved by a method for the recycling of a textile waste comprising a cellulosic component and a polyester component, comprising the following steps:
a depolymerization step, comprising submitting the textile waste to a depolymerization reaction of the polyester component by basic hydrolysis by means of a solvolytic mixture comprising an alkali metal hydroxide and at least one water-soluble organic reaction solvent, until obtaining a biphasic mixture comprising an initial solid phase, containing a terephthalic acid salt and cellulosic fibres, and an initial liquid phase, containing the water-soluble organic reaction solvent;
a first step of solid-liquid separation, comprising the removal of at least a part of the initial liquid phase from the biphasic mixture;
a step of treatment with organic solvent, wherein following the first step of solid-liquid separation, the initial solid phase is treated with an organic washing solvent until removing, from the initial solid phase, one or more possible contaminants present therein, and obtaining a solid purified phase containing the terephthalic acid salt and the cellulosic fibres;
a second step of solid-liquid separation, wherein the solid purified phase is separated from the organic washing solvent and from the one or more possible contaminants dissolved therein;
a step of treatment with water, comprising bringing the solid purified phase into contact with washing water until solubilizing the terephthalic acid salt contained therein, and obtaining, by the removal of the later, a final solid phase comprising the cellulosic fibres.

Advantageously, the solvolytic mixture used in the basic hydrolysis process may also comprise reaction water.

Preferably, the organic washing solvent is or comprises one of the following substances:
a glycol having two to eight carbon atoms (C₂-C₈);
an alcohol C₁-C₆ or a mixture of alcohols C₁-C₆;
a ketone C₃-C₈ or a mixture of ketones C₃-C₈;
an ether or a mixture of ethers;
an alkane C₅-C₈ or a mixture of alkanes C₅-C₈.

Preferably, the glycol is a glycol having a boiling temperature of less than 300° C.

Preferably, the glycol is chosen from: monoethylene glycol (MEG), diethylene glycol (DEG), triethylene glycol (TEG) and mixtures thereof; even more preferably, the glycol is monoethylene glycol (MEG).

Preferably, the alcohol C₁-C₆ is chosen from: methanol (MeOH), ethanol (EtOH), isopropanol (i-PrOH), butanol (BuOH) and mixtures thereof.

Preferably, the ketone C₃-C₈ is chosen from: acetone, methyl ethyl ketone (MEK), methyl isobutyl ketone (MIBK) and mixtures thereof.

Preferably, the ether is chosen from: diethylether (Et₂O), tetrahydrofuran (THF) and mixtures thereof.

Preferably, the alkane C₅-C₈ is chosen from: hexane, heptane, octane and mixtures thereof.

Advantageously, the water-soluble organic reaction solvent and the organic washing solvent are or comprise the same substance.

Preferably, the alkali metal hydroxide is chosen from: sodium hydroxide (NaOH), potassium hydroxide (KOH), lithium hydroxide (LiOH) and mixtures thereof.

In an advantageous embodiment, the depolymerization reaction by basic hydrolysis is microwave-assisted.

Advantageously, the method according to the invention comprises, prior to the depolymerization step, a step of preparing the textile waste comprising shredding the textile waste.

Preferably, the second step of solid-liquid separation comprises separating the solid purified phase from the organic washing solvent and from the one or more possible contaminants dissolved therein, by evaporation.

Advantageously, the second step of solid-liquid separation comprises separating the solid purified phase from the organic washing solvent and from the one or more possible contaminants dissolved therein, by evaporation, at a pressure below the atmospheric pressure.

Advantageously, the organic washing solvent used in the step of treatment with organic solvent comprises a portion of fresh organic washing solvent and a portion of recovered organic washing solvent, obtained by separating the same from a solid purified phase in a second step of solid-liquid separation of a method according to the invention, performed prior to the step of treatment with organic solvent.

Advantageously, the step of treatment with organic solvent comprises one or more sub-steps of solid-solvent mixing, wherein the initial solid phase is added to and mixed with the organic washing solvent, each followed by a sub-step of solid-solvent separation, wherein the organic washing solvent is removed, at least partially, from the initial solid phase.

Preferably, the one or more sub-steps of solid-solvent mixing comprise two or more sub-steps of solid-solvent mixing and the purity of the organic washing solvent employed in the two or more sub-steps of solid-solvent mixing advantageously increases from the first to the last of the two or more sub-steps of solid-solvent mixing.

Advantageously, the organic washing solvent used in the last of the one or more sub-steps of solid-solvent mixing comprises a portion of fresh organic washing solvent and a portion of recovered organic washing solvent obtained by removing it from a solid purified phase in a second step of solid-liquid separation of a method according to the invention, performed prior to said last of said one or more sub-steps of solid-solvent mixing.

Advantageously, the method according to the invention comprises, after the step of treatment with water, a step of water removal comprising removing, from the final solid phase, any washing water present therein.

Preferably, the step of water removal comprises removing, from the final solid phase, any washing water present therein, by evaporation.

Advantageously, the step of water removal comprises removing, from the final solid phase, any washing water present therein, by evaporation, at a pressure below the atmospheric pressure.

Advantageously, the washing water used in the step of treatment with water may comprise a portion of fresh washing water and a portion of recovered washing water, removed from a final solid phase in a step of water removal of a method for the recycling of a textile waste according to the invention, performed previously.

The step of washing with water preferably comprises one or more sub-steps of solid-water mixing, wherein the solid purified phase is added to and mixed with water, each followed by a sub-step of solid-water separation, wherein the water is removed, at least partially, from the solid purified phase.

The one or more sub-steps of solid-water mixing advantageously comprise two or more sub-steps of solid-water mixing and the purity of the washing water employed in the one or more sub-steps of solid-water mixing increases from the first to the last of the one or more sub-steps of solid-water mixing.

Advantageously, the water used in the last of the one or more sub-steps of solid-water mixing comprises a portion of fresh washing water and a portion of water removed from a final solid phase in a step of water removal of a method for the recycling of a textile waste according to the invention, performed prior to the last of the one or more sub-steps of solid-water mixing.

In an advantageous embodiment, in the step of treatment with water, the washing water is at least partially removed from the solid purified phase and the washing water thus removed is submitted to a step of precipitation of the terephthalic acid wherein it is reacted with a mineral acid to form a suspension of initial terephthalic acid in an acid aqueous solution, the method comprising a step of separation of the terephthalic acid, wherein said initial terephthalic acid is removed from the acid aqueous solution, said acid aqueous solution then being submitted to a step of neutralization of the method, wherein the acid aqueous solution is brought into contact with an alkaline base to form an alkali metal salt and obtain a neutral aqueous solution, the method comprising submitting the neutral aqueous solution to a step of recovery, comprising a step of electrolysis and an optional step of combination, from which the same mineral acid used in the step of precipitation of the terephthalic acid and the same alkaline base used in the step of neutralization, are obtained, the mineral acid thus obtained being used in a further step of precipitation of the terephthalic acid of a method according to the invention, performed subsequently.

The features and advantages of this invention will be more evident from the following description, which is illustrative and not limiting, referring to the attached schematic drawings, in which:

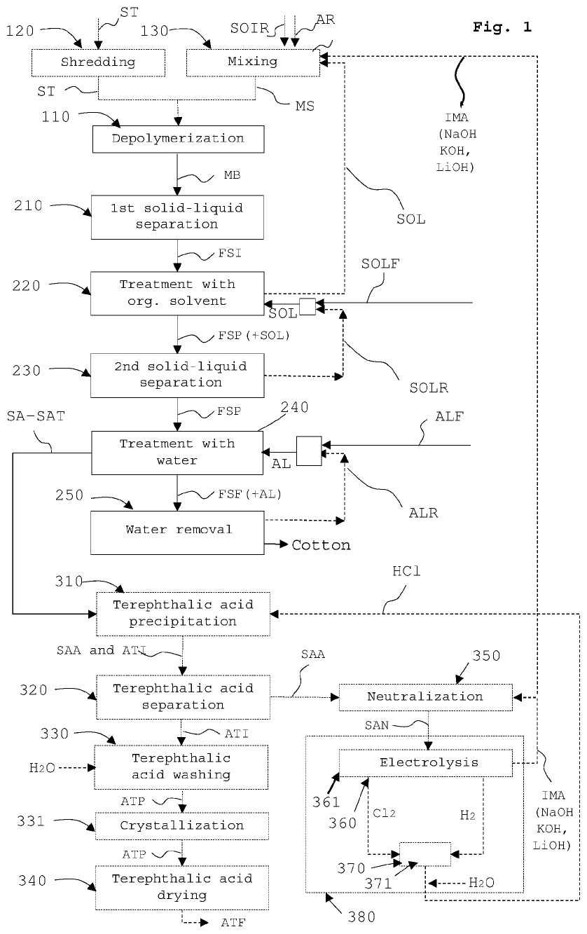

FIG. 1 is a flow chart that schematically represents the steps of an advantageous implementation of method according to the invention;

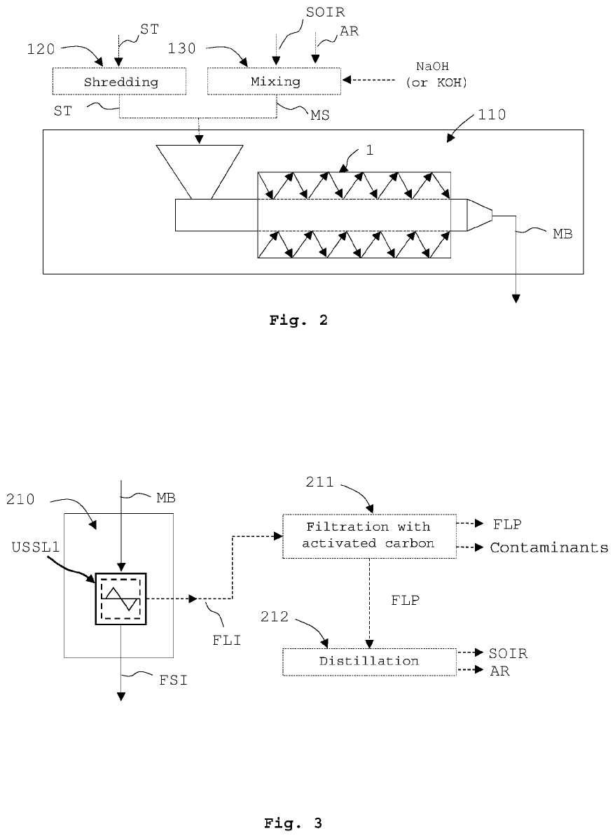

FIG. 2 is a more detailed schematic representation of one of the steps in the method in FIG. 1;

FIG. 3 is a more detailed schematic representation of one step of the method in FIG. 1;

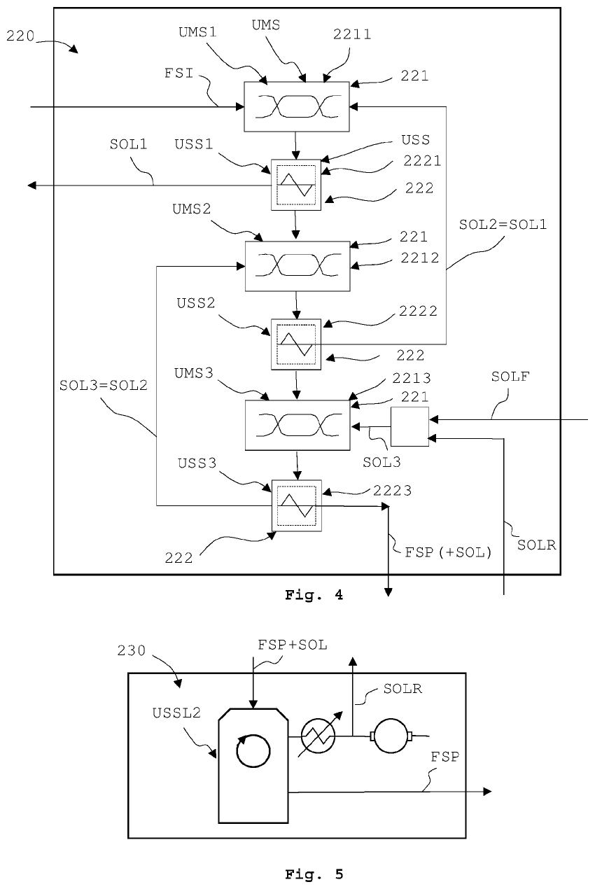

FIG. 4 is a more detailed schematic representation of one step of the method in FIG. 1;

FIG. 5 is a more detailed schematic representation of one step of the method in FIG. 1;

FIG. 6 is a more detailed schematic representation of one step of the method in FIG. 1;

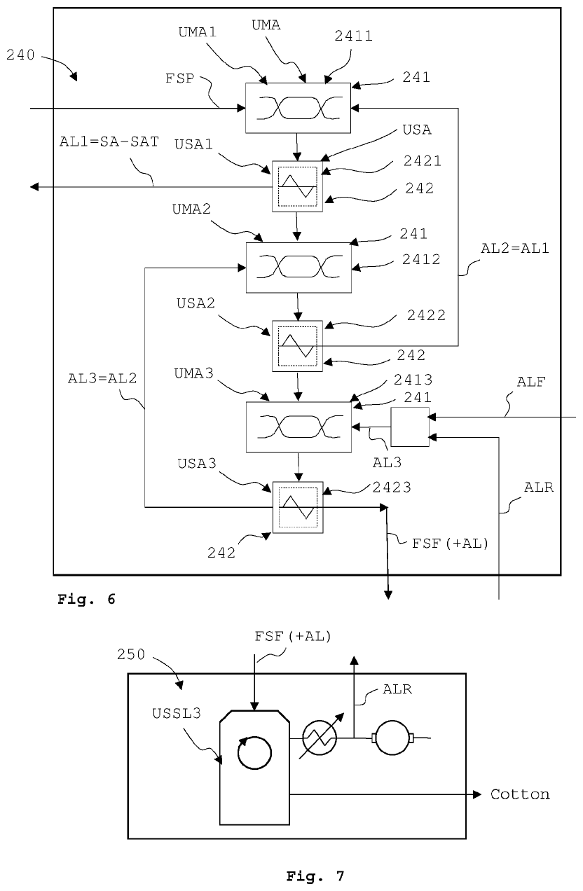

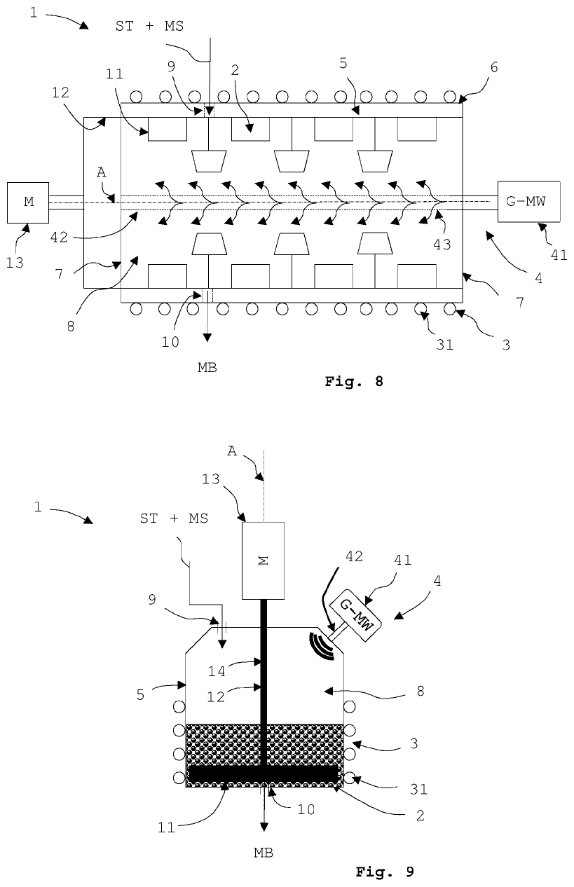

FIG. 7 is a more detailed schematic representation of one step of the method in FIG. 1;

FIG. 8 is a schematic representation of an advantageous embodiment of a reactor which is usable in a method according to the invention;

FIG. 9 is a schematic representation of a further advantageous embodiment of a reactor which is usable in a method according to the invention;

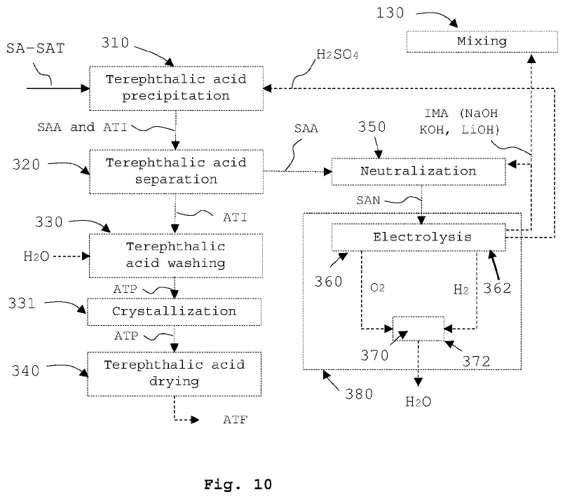

FIG. 10 is a detail of a flow chart schematically illustrating some steps of a further advantageous implementation of method according to the invention.

FIG. 1 shows a flow chart schematically illustrating the various steps of an example of an advantageous implementation of method according to the invention.

The method according to the invention includes a depolymerization step 110 in which a textile waste ST, comprising a cellulosic component, e.g. cotton, and a polyester component, is submitted to a depolymerization reaction of the polyester component by basic hydrolysis; hereafter, this reaction will be referred to as the “depolymerization reaction”.

The percentages of cellulosic component and polyester component in the textile waste ST may vary, but preferably the cellulosic component (e.g. cotton) is between 15% and 65% by weight of the textile waste ST.

The depolymerization reaction occurs by contact of the textile waste ST with a solvolytic mixture MS comprising possible reaction water AR, an alkali metal hydroxide IMA, and a water-soluble organic reaction solvent SOIR; the alkali metal hydroxide IMA may comprise, for example sodium hydroxide NaOH and/or potassium hydroxide KOH and/or lithium hydroxide LiOH; with regard to the water-soluble organic reaction solvent SOIR, examples will be provided below.

The depolymerization reaction is preferably microwave-assisted and is advantageously conducted into a reactor 1, preferably of the microwave type, shown schematically in FIG. 2.

An advantageous example of a microwave reactor 1 which is usable in the method according to the invention is illustrated, for example, in European Patent EP 2 736 968 B1 in the name of the same Applicant as this application.

A further example of an advantageous embodiment of reactor 1 is illustrated schematically in FIG. 8.

Such a reactor 1 advantageously comprises a mixing device 2, a thermal control system 3 and a microwave generation and transport system 4.

Advantageously, the reactor 1 comprises a casing 5 which delimits therein a reaction chamber 8; said casing 5, preferably substantially cylindrical in shape, extends along and around a longitudinal axis A which, in use, is substantially horizontal.

It is understood that in use, the casing 5, and consequently the reactor 1 itself, may be differently oriented, and that instead of horizontal, the axis A may therefore be vertical or even variously inclined with respect to a horizontal plane (e.g. with respect to the ground).

Advantageously, the casing 5 comprises a side wall 6, e.g. cylindrical, and a pair of end walls 7 positioned at opposite axial ends that internally delimit the reaction chamber 8.

The casing 5 is advantageously constructed of a material that is substantially opaque to microwave electromagnetic radiation and preferably is corrosion resistant.

In addition, the casing 5 is advantageously designed to be able to withstand both internal overpressure and underpressure conditions relative to atmospheric pressure.

The reactor 1 is advantageously provided with one or more inlets and one or more outlets defined by respective openings preferably formed in the casing 5 and configured to allow, respectively, the introduction into the reaction chamber 8 of the various components participating in the depolymerization reaction such as, for example the textile waste ST, the solvolytic mixture MS and possible auxiliary components, and the removal from the reaction chamber 8 of the products of said depolymerization reaction and possible residual elements.

In an advantageous embodiment, such as the one shown in FIG. 8, the reactor 1 is provided with at least one inlet 9 for introducing the material to be treated in the reactor 1 (e.g. the textile waste ST, possibly shredded), the solvolytic mixture MS and possible auxiliary components, into the reaction chamber 8.

In a further advantageous embodiment (not shown), the material to be treated, the solvolytic mixture and possible auxiliary components may be introduced into the reactor 1 through dedicated, separate inlets.

Advantageously, the reactor 1 comprises at least one outlet 10 for removing the contents of the reaction chamber 8 at the end of the depolymerization reaction.

Advantageously, the reaction chamber 8 houses the mixing device 2, which is rotatable around the axis A.

The mixing device 2 is advantageously configured to keep agitated—through its rotational motion around the axis A—the contents of the reaction chamber 8, comprising, for example the textile waste ST and the solvolytic mixture MS, by distributing it dynamically within the same, both along the axis A and radially with respect to the axis A.

Preferably, the mixing device 2 includes a plurality of paddles 11, which may be variously shaped, sized and arranged.

In an advantageous embodiment, the paddles 11 are solidly associated with a support structure 12 housed in the reaction chamber 8 and rotatable around the axis A.

For example, the support structure 12 may advantageously include a hollow cylindrical basket housed in the casing 5 and having a radially outer side surface facing the side wall 6 of the casing 5 and a radially inner side surface from which the paddles 11 extend radially toward the axis A.

Advantageously, the paddles 11 may have different shapes and sizes and be variously placed on the support structure 12.

It is understood however, that the mixing device 2 may be of other types than those shown herein by way of example only, and include variously located paddles having various shapes and sizes.

In an example of advantageous embodiment of reactor 1 (not illustrated in the accompanying drawings), the variously shaped paddles 11, also ribbon-shaped paddles, may advantageously depart from a support structure 12 centrally located in the reaction chamber 8 along the axis A.

In other advantageous embodiments (not shown), the mixing device 2 may be integral with the casing 5 and rotate integrally therewith around the axis A; in such advantageous embodiments, the casing 5 is then rotatably mounted on an external support (not shown) and rotatably drags the paddles 11.

The mixing device 2 is advantageously driven by a motor 13 located, for example outside the casing 5, and preferably connected to the support structure 12 (or advantageously directly to the casing 5, if this is rotatable).

Advantageously, the thermal control system 3 is configured to extract or introduce thermal energy (heat) from/into the reaction chamber 8 in the casing 5 and thereby cool or heat the casing 5 and whatever is contained therein.

Advantageously, the thermal control system 3 comprises at least one coil 31 wound around the side wall 6 of the casing 5, within which a heat transfer fluid circulates which is suitable for transmitting the heat preferably from a thermal unit (not illustrated) where said fluid is appropriately heated or cooled.

Advantageously, the microwave generation and transport system 4 comprises a microwave generator 41, preferably located outside the casing 5, and a guiding device 42, associated with the microwave generator 41 and housed at least partially in the reaction 8, advantageously configured to carry and distribute the microwaves generated by the microwave generator 41 in the reaction chamber 8, along and around the axis A.

Advantageously, the guiding device 42 comprises a waveguide 43 (of the so-called “leaking pipe” type) comprising a pipe extending along the axis A of the casing 5, between the pair of end walls 10, and advantageously having side emission holes formed on a side wall of the pipe.

Advantageously, microwaves with frequencies between 300 MHz and 300 GHz are sent into the reaction chamber 8.

In a further advantageous example of an embodiment of reactor 1 (such as that illustrated schematically in FIG. 9), the reactor 1 also comprises a mixing device 2, a thermal control system 3 and a microwave generation and transport system 4.

Compared to the advantageous embodiment illustrated in FIG. 8, the reactor 1 and the casing 5 in the advantageous embodiment illustrated in FIG. 9 extend along and around a longitudinal axis A which, in use, is substantially vertical; the axis A is also the axis of rotation of the mixing device 2.

Here again, the mixing device 2 is advantageously configured to keep the contents of the casing 5 (e.g. the textile waste ST and the solvolytic mixture MS) agitated through its own rotational motion by distributing it dynamically in the reaction chamber 8, both along the axis A and radially with respect to the axis A.

Advantageously, the mixing device 3, advantageously driven by the motor 13, is configured such that the support structure 12, comprising at least one impeller 14 and at least one paddle 11, is not only rotatable around the axis A, but also axially movable along the axis A.

Advantageously, the mixing device 2 may take on various configurations, for example it may provide one or more variously shaped and organized paddles 11 possibly placed on more impellers 14.

Also in the example of advantageous embodiment of reactor 1 shown in FIG. 9, the reactor 1, and possible contents thereof, may be advantageously cooled or heated thanks to a thermal control system 3.

Advantageously, the thermal control system 3, either in the advantageous embodiment illustrated for example in FIG. 8 or in the one illustrated in FIG. 9, may also include, in addition to or as an alternative to the coil 31, a further heating/cooling device (not shown in the accompanying drawings) acting directly on the mixing device 2, for example on the paddles 11 and/or the impeller 14.

Advantageously, if provided, the microwave generation and transport system 4 can directly introduce the microwaves required by the depolymerization reaction into the reaction chamber 8, such as through a waveguide 42.

Advantageously, the method according to the invention comprises, prior to the depolymerization step 110, a mixing step 130 in which possible reaction water AR, the water-soluble organic reaction solvent SOIR and the alkali metal hydroxide IMA are mixed to form the solvolytic mixture MS.

Preferably, the method according to the invention further comprises, prior to the depolymerization step 110, a shredding step 120 of the textile waste ST, wherein said waste is shredded into small pieces.

Advantageously, the reactor 1 is then fed with the solvolytic mixture MS and the textile waste ST, which is preferably shredded; the depolymerization reaction of the polyester component proceeds in the reactor 1 until a biphasic mixture MB is obtained.

Advantageously, the depolymerization reaction is conducted at a temperature in the range of 90° C. to 350° C., preferably between 100° C. and 200° C.

Advantageously, the depolymerization reaction is conducted at a pressure in the range of 1 atm to 20 atm.

The biphasic mixture MB comprises an initial solid phase FSI containing a terephthalic acid salt SAT and cellulosic fibres, e.g. cotton fibres, and an initial liquid phase FLI.

The initial liquid phase FLI comprises water-soluble organic reaction solvent SOIR, a possible residual fraction of the reaction water AR, possible dissolved contaminants, such as dyes and/or a possible fraction of the alkali metal hydroxide IMA that did not participate in the depolymerization reaction, and a possible dissolved fraction of the terephthalic acid salt SAT.

An undissolved fraction of the terephthalic acid salt SAT occurs as solid particles that can be freely suspended in the initial liquid phase FLI and/or trapped between the cellulosic fibres which are soaked in the initial liquid phase FLI.

The method according to the invention comprises, after the depolymerization step 110, a first step of solid-liquid separation 210 in which the removal of at least part of the initial liquid phase FLI from the biphasic mixture MB takes place; this removal preferably takes place by mechanical filtration, for example in a first solid-liquid separation unit USSL1 (schematically shown in FIG. 3).

The solid-liquid separation unit USSL1 can be, for example an industrial machine of the type commonly known as screw filter.

Advantageously, the initial liquid phase FLI, removed during the first step of solid-liquid separation 210, can be submitted to a step of filtration with activated carbon 211 (illustrated schematically in FIG. 3), in which any contaminants are removed, thus obtaining a purified liquid phase FLP, which can advantageously be submitted to a subsequent distillation step 212, in which the reaction water AR and the water-soluble organic reaction solvent SOIR contained in said purified liquid phase FLP are separated by distillation to be possibly reused later, for example in a depolymerization step 110 performed subsequently.

In a further advantageous embodiment (not illustrated), the initial liquid phase FLI removed during the first step of solid-liquid separation 210 may be employed, either directly or in the form of a purified liquid phase FLP (after being submitted to a possible step of filtration with activated carbon 211), in a depolymerization step 210 performed subsequently, without being submitted to the distillation step 212.

It is emphasized that although much of the initial liquid phase FLI of the biphasic mixture MB is removed during the first step of solid-liquid separation 210, the initial solid phase FSI exiting the first step of solid-liquid separation 210 could possibly contain—for example trapped between the cellulosic fibres—a residual fraction of the initial liquid phase FLI, comprising the one or more contaminants dissolved therein.

Therefore, the method according to the invention comprises, after the first step of solid-liquid separation 210, a step of treatment with organic solvent 220, wherein the initial solid phase FSI, comprising the cellulosic fibres (e.g. cotton), a terephthalic acid salt SAT and a possible residual fraction of the initial liquid phase FLI, comprising the one or more contaminants, if any, dissolved therein, is treated with an organic washing solvent SOL in order to remove the one or more possible contaminants present therein.

Advantageously, the organic washing solvent SOL is, or comprises, one of the following substances:
a glycol C₂-C₈;
an alcohol C₁-C₆ or a mixture of alcohols C₁-C₆;
a ketone C₃-C₈ or a mixture of ketones C₃-C₈;
an ether or a mixture of ethers;
an alkane C₅-C₈ or a mixture of alkanes C₅-C₈.

Preferably, the glycol is a glycol having a boiling temperature of less than 300° C.

Preferably, the glycol is chosen from: monoethylene glycol (MEG), diethylene glycol (DEG), triethylene glycol (TEG) and mixtures thereof; even more preferably, the glycol is monoethylene glycol (MEG).

Preferably, the alcohol C₁-C₆ is chosen from: methanol (MeOH), ethanol (EtOH), isopropanol (i-PrOH), butanol (BuOH) and mixtures thereof.

Preferably, the ketone C₃-C₈ is chosen from: acetone, methyl ethyl ketone (MEK), methyl isobutyl ketone (MIBK) and mixtures thereof.

Preferably, the ether is chosen from: diethylether (Et₂O), tetrahydrofuran (THF) and mixtures thereof.

Preferably, the alkane C₅-C₈ is chosen from: hexane, heptane, octane and mixtures thereof.

Preferably, the water-soluble organic reaction solvent SOIR and the organic washing solvent SOL are the same substance or the same mixture of substances.

At the end of the step of treatment with organic solvent 220, a solid purified phase FSP is obtained comprising the terephthalic acid salt SAT and cellulosic fibres, e.g. cotton fibres, possibly still soaked in a given amount of organic washing solvent SOL and possible contaminants dissolved therein.

Advantageously, the method according to the invention comprises, after the step of treatment with organic solvent 220, a second step of solid-liquid separation 230 in which the solid purified phase FSP is separated from possible organic washing solvent SOL still present, and from the one or more possible contaminants dissolved therein.

Such separation is preferably performed by evaporation, advantageously in a second solid-liquid separation unit USSL2 (illustrated schematically in FIG. 5); the evaporation is preferably performed at a pressure below atmospheric pressure.

Advantageously, the organic washing solvent SOL used in the step of treatment with organic solvent 220 may comprise a portion of fresh organic washing solvent SOLF, and a portion of recovered organic washing solvent SOLR, obtained by separating the same from a solid purified phase FSP in a second step of solid-liquid separation 230, preferably by evaporation and subsequent condensation, performed prior to the step of treatment with organic solvent 220.

Advantageously, the step of treatment with organic solvent 220 may comprise one or more sub-steps of solid-solvent mixing 221, wherein the initial solid phase FSI, following the first step of solid-liquid separation 210, is added to and mixed with the organic washing solvent SOL; such one or more sub-steps of solid-solvent mixing 221 are preferably performed in one or more solvent mixing units UMS.

The solvent mixing unit UMS is preferably an industrial machine of the type commonly known as ribbon mixer.

Advantageously, the organic washing solvent SOL employed in the one or more sub-steps of solid-solvent mixing 221 comprises at least a part of the water-soluble organic reaction solvent SOIR distilled in a possible distillation step 212 performed previously.

Each of such one or more sub-steps of solid-solvent mixing 221 is preferably followed by a respective sub-step of solid-solvent separation 222, wherein the organic washing solvent SOL, submitted to said sub-step of solid-solvent mixing 221, is at least partially removed from the initial solid phase FSI, preferably by mechanical filtration in one or more solvent separation units USS.

The solvent separation unit USS is preferably an industrial machine of the type commonly known as screw filter.

Advantageously, as will be better explained below, the one or more sub-steps of solid-solvent mixing 221 comprise two or more sub-steps of solid-solvent mixing 221; preferably, the purity of the organic washing solvent SOL, employed in said two or more sub-steps of solid-solvent mixing 221, increases from the first to the last of said two or more sub-steps.

The organic washing solvent SOL used in the last of the one or more sub-steps of solid-solvent mixing 221 preferably comprises a portion of fresh organic washing solvent SOLF, and a portion of recovered organic washing solvent SOLR, obtained by removing the same from a solid purified phase FSP in a second step of solid-liquid separation 230, preferably by evaporation and subsequent condensation, performed prior to the last of the one or more sub-steps of solid-solvent mixing 221.

FIG. 4 schematically depicts an example of step of treatment with organic solvent 220 of an advantageous embodiment of the method according to the invention.

In such an advantageous embodiment, the step of treatment with organic solvent 220 comprises three sub-steps of solid-solvent mixing 221, each followed by a respective sub-step of solid-solvent separation 222, in series with each other.

In a first sub-step of solid-solvent mixing 2211, advantageously performed in a first solvent mixing unit UMS1, the initial solid phase FSI is, for the first time, added to and mixed with organic washing solvent; this solvent is herein called organic solvent of first washing SOL1.

Advantageously, after the first sub-step of solid-solvent mixing 2211, the organic solvent of first washing SOL1 is at least partially removed from the initial solid phase FSI in a first sub-step of solid-solvent separation 2221, advantageously performed in a first solvent separation unit USS1.

Advantageously, the organic solvent of first washing SOL1, removed during the first sub-step of solid-solvent separation 2221, can be at least partially reused, for example in a depolymerization step 110 performed subsequently, preferably after a filtration with activated carbon, for example to integrate the water-soluble organic reaction solvent SOIR in the solvolytic mixture MS employed in such depolymerization reaction.

After the first sub-step of solid-solvent separation 2221, the initial solid phase FSI is added to and mixed with organic washing solvent a second time in a second sub-step of solid-solvent mixing 2212, advantageously performed in a second solvent mixing unit UMS2; said solvent is herein called organic solvent of second washing SOL2.

Advantageously, after the second sub-step of solid-solvent mixing 2212, the organic solvent of second washing SOL2 is at least partially removed from the initial solid phase FSI in a second sub-step of solid-solvent separation 2222, advantageously performed in a first solvent separation unit USS2.

Advantageously, the organic solvent of second washing SOL2, separated from the initial solid phase FSI in the second solvent separation unit USS2, can be at least partially reused as the solvent of first washing SOL1 in a further first sub-step of solid-solvent mixing 2211 performed subsequently.

After the second sub-step of solid-solvent separation 2222, the initial solid phase FSI is added to and mixed with organic solvent of third washing SOL3, preferably comprising fresh organic washing solvent SOLF, in a third sub-step of solid-solvent mixing 2213, advantageously performed in a third solvent mixing unit UMS3.

Advantageously, the organic solvent of third washing SOL3 may comprise a recovered organic washing solvent SOLR, obtained by separating the same from a solid purified phase FSP in a second step of solid-liquid separation 230, preferably by evaporation and subsequent condensation, performed prior to the third sub-step of solid-solvent mixing 2213.

Advantageously, after the third sub-step of solid-solvent mixing 2213, the organic solvent of third washing SOL3 is at least partially removed from the initial solid phase FSI in a third sub-step of solid-solvent separation 2223, advantageously performed in a third solvent separation unit USS3, thus obtaining the solid purified phase FSP, optionally soaked in the organic solvent of third washing SOL3.

Advantageously, the organic solvent of third washing SOL3, separated from the initial solid phase FSI in the third solvent separation unit USS3, can be at least partially reused as the solvent of second washing SOL2 in an further second sub-step of solid-solvent mixing 2212 performed subsequently.

In other words, in this advantageous embodiment, the organic washing solvent SOL is reused several times, at a progressively lower level of purity, in sub-steps of solid-solvent mixing performed at different times; a certain sub-step of solid-solvent mixing is thus performed with a purer organic washing solvent than that employed in a preceding sub-step of solid-solvent mixing.

The method according to the invention comprises, after the second step of solid-liquid separation 230, a step of treatment with water 240, during which the solid purified phase FSP is brought into contact with washing water AL in order to solubilize and remove the terephthalic acid salt SAT present, thus obtaining a final solid phase FSF comprising cellulosic fibres, for example cotton fibres, possibly still soaked in a minimal amount of washing water AL.

The solubilization of the terephthalic acid salt SAT in the washing water AL is possible due to the good solubility of the same in water, which is equal, for example, to about 13 wt % in the case where the terephthalic acid salt SAT is a sodium-based salt, such as sodium terephthalate Na₂TA; such solubilization allows the removal of solid particles of the terephthalic acid salt SAT possibly trapped between the cellulosic fibres.

Advantageously, during the step of treatment with water 240, the washing water AL is at least partially removed from the solid purified phase FSP; this removed washing water AL, which is indicated in FIG. 1 as SA-SAT, which in fact is an aqueous solution comprising the terephthalic acid salt SAT solubilized in water, can advantageously be submitted, possibly after being submitted to one or more further filtration steps (for example, mechanical filtration and/or filtration with activated carbon) (which are not shown) to remove possible contaminants still present, to a possible step of precipitation of the terephthalic acid 310 wherein a mineral acid is added, for example hydrochloric acid HCl or sulfuric acid H₂SO₄, thus obtaining a suspension of initial terephthalic acid ATI (solid) in an acid aqueous solution SAA of the salt respective to the acid used and the base used in the depolymerization reaction (for example NaCl).

Advantageously, the method according to the invention comprises, after the step of treatment with water 240, a third solid-liquid separation step 250, wherein the final solid phase FSF is separated from possible washing water AL still present and from the one or more possible contaminants dissolved therein; preferably, the separation occurs by evaporation, more preferably in a third solid-liquid separation unit USSL3 (illustrated schematically in FIG. 7).

The evaporation is preferably performed at a pressure below atmospheric pressure.

At the end of the third solid-liquid separation step 250, the cellulosic component, e.g. the cotton fibres that have been separated from the polyester and from possible contaminants in the textile waste ST, can be used, for example to make a new fabric.

Advantageously, the washing water AL used in the step of treatment with water 240, may comprise a portion of fresh washing water ALF and a portion of recovered washing water ALR, removed from a final solid phase FSF in a step of water removal 250 of a method for the recycling of a textile waste according to the invention, performed previously.

Advantageously, the step of treatment with water 240 may comprise one or more sub-steps of solid-water mixing 241, wherein the solid purified phase FSP is added to and mixed with washing water AL; such one or more sub-steps of solid-water mixing 241 are preferably performed in one or more water mixing units UMA.

The water mixing unit UMA is preferably an industrial machine of the type commonly known as ribbon mixer.

Advantageously, the washing water AL employed in the one or more of the sub-steps of solid-water mixing 241 may comprise at least a part of the reaction water AR distilled in a distillation step 212 performed previously.

Each of the one or more sub-steps of solid-water mixing 241 is preferably followed by a sub-step of solid-water separation 242, wherein the washing water AL is at least partially removed from the solid purified phase FSP, preferably by mechanical filtration in one or more water separation units USA.

The water separation unit USA is preferably an industrial machine of the type commonly known as screw filter.

Advantageously, the one or more sub-steps of solid-water mixing 241 comprise two or more sub-steps of solid-water mixing 241 and, as will be explained in more detail below, the purity of the washing water AL employed in the one or more sub-steps of solid-water mixing 241 increases from the first to the last of such one or more sub-steps of solid-water mixing 241.

Advantageously, the water used in the last of the one or more sub-steps of solid-water mixing 241 may comprise a portion of fresh washing water ALF and a portion of recovered washing water ALR removed from a final solid phase FSF in a step of water removal 250, preferably by evaporation and subsequent condensation of any remaining washing water AL, of a method for the recycling of a textile waste according to the invention, performed prior to the last of the one or more sub-steps of solid-water mixing 241.

FIG. 6 schematically depicts an example of a step of treatment with water 240 of an advantageous embodiment of the method according to the invention.

In such an advantageous embodiment, the step of treatment with water 240 comprises three sub-steps of solid-water mixing 241, each followed by a respective sub-step of solid-water separation 242, in series with each other.

In a first sub-step of solid-water mixing 2411, advantageously performed in a first water mixing unit UMA1, the solid purified phase FSP is, for the first time, added to and mixed with washing water; such water is herein called first washing water AL1.

Advantageously, after the first sub-step of solid-water mixing 2411, the first washing water AL1 is at least partially removed from the solid purified phase FSP in a first sub-step of solid-water separation 2421, advantageously performed in a first water separation unit USA1; the first washing water AL1 thus removed (shown in FIG. 6 as SA-SAT) is in fact an aqueous solution comprising the solubilized terephthalic acid salt.

In a second sub-step of solid-water mixing 2412, advantageously performed in a second water mixing unit UMA2, subsequent to the first sub-step of solid-water separation 2421, the solid purified phase FSP is added to and mixed a second time with washing water; such water is herein called second washing water AL2.

Advantageously, after the second sub-step of solid-water mixing 2412, the second washing water AL2 is removed at least in part from the solid purified phase FSP in a second sub-step of solid-water separation 2422, advantageously performed in a second water separation unit USA2.

Advantageously, the second washing water AL2, separated from the solid purified phase FSP in the second water separation unit USA2, can be at least partially reused as first washing water ALL in an additional first sub-step of solid-water mixing 2411 performed subsequently.

In a third sub-step of solid-water mixing 2413, advantageously performed in a third water mixing unit UMA3, subsequent to the second sub-step of solid-water separation 2422, the solid purified phase FSP is added to and mixed a second time with third washing water AL3, preferably comprising fresh washing water ALF.

Advantageously, the third washing water AL3 may comprise recovered washing water ALR removed from a final solid phase FSF in a step of water removal 250 of a method for the recycling of a textile waste according to the invention, performed previously.

Advantageously, after the third sub-step of solid-water mixing 2413, the third washing water AL3 is at least partially removed from the solid purified phase FSP in a third sub-step solid-water separation 2423, advantageously performed in a third water separation unit USA3, thus obtaining the final solid phase FSF, optionally soaked in the residual third washing water AL3.

Advantageously, the third washing water AL3, separated from the solid purified phase FSP in the third water separation unit USA3, can be at least partially reused as second washing water AL2 in a further second sub-step of solid-water mixing 2412 performed subsequently.

In other words, in this advantageous embodiment, the washing water AL is reused several times, at a progressively lower level of purity, in sub-steps of solid-water mixing performed at different times; a certain sub-step of solid-water mixing is thus performed with a purer washing water than that employed in a preceding sub-step of solid-water mixing.

As mentioned, the aqueous solution of a terephthalic acid salt SA-SAT removed in the step of solid-water separation 240 (which in the advantageous embodiment illustrated in FIG. 6 is the first washing water AL1 separated from the solid purified phase FSP in the first solid-water separation unit 2421) can be advantageously submitted to the step of precipitation of the terephthalic acid 310, wherein a mineral acid is added, for example hydrochloric acid (HCl) or sulfuric acid (H₂SO₄), thus obtaining a suspension of initial terephthalic acid ATI (solid) in an acid aqueous solution SAA of the salt respective to the acid used (for example sodium chloride (NaCl) in the case (HCl) is employed, or for example sodium sulphate (Na₂SO₄) in the case sulfuric acid (H₂SO₄) is used) and of the base used in the depolymerization reaction (for example, alkali metal hydroxide IMA, such as for example sodium hydroxide (NaOH) and/or potassium hydroxide (KOH) and/or lithium hydroxide (LiOH)).

In addition, in the step of precipitation of the terephthalic acid 310, the suspended initial terephthalic acid ATI separates from the acid aqueous solution SAA by precipitation.

Advantageously, after the step of precipitation of the terephthalic acid 310, in a step of separation of the terephthalic acid 320, the acid aqueous solution SAA is at least partially removed from the precipitated initial terephthalic acid ATI, preferably by mechanical filtration, for example by centrifugation.

Preferably, after said step of separation of the terephthalic acid 320, the initial terephthalic acid ATI, possibly still wetted by a fraction of the acid aqueous solution SAA, is submitted to a step of washing of the terephthalic acid 330, wherein it is placed in contact with water, preferably fresh water, for the removal of possible residual mineral acid remaining from the previous steps of precipitation of the terephthalic acid 310 and of separation of the terephthalic acid 320, thus obtaining purified terephthalic acid ATP.

Preferably, after the step of washing the terephthalic acid 330, the purified terephthalic acid ATP is submitted to a step of drying the terephthalic acid 340, wherein the water employed during the step of washing the terephthalic acid 330 with water is removed from the purified terephthalic acid ATP, preferably by evaporation, thus obtaining the final terephthalic acid ATF.

The purity of the final terephthalic acid ATF thus obtained is such that it can be used in a new polymerization reaction, for example to produce PET.

Advantageously, between the step of washing the terephthalic acid 330 and the step of drying the terephthalic acid 340, a step of crystallization 331 of the terephthalic acid may be provided, wherein the latter is submitted to crystallization.

Advantageously, the acid aqueous solution SAA, separated from the initial terephthalic acid ATI during the step of separation of the terephthalic acid 320, may be submitted to a step of neutralization 350, wherein it is placed in contact with one or more alkali bases, such as an alkali metal hydroxide IMA such as, for example, sodium hydroxide (NaOH) and/or potassium hydroxide (KOH) and/or lithium hydroxide (LiOH).

The reaction of such one or more alkali bases with the mineral acid, e.g. hydrochloric acid (HCl) or sulfuric acid (H₂SO₄), possibly still present in the acid aqueous solution SAA, leads to the formation of alkali metal salts (e.g. sodium chloride (NaCl) and/or potassium chloride (KCl) and/or lithium chloride (LiCl), in the case where hydrochloric acid (HCl) is used in the step of precipitation of the terephthalic acid 310, or sodium sulphate (Na₂SO₄), in the case where sulfuric acid (H₂SO₄) is used), and to the consequent neutralization of the pH of the acidic aqueous solution SAA, thus obtaining a neutral aqueous solution SAN having a pH substantially equal to 7.

Advantageously, such neutral aqueous solution SAN, containing an alkali metal salt (for example, sodium chloride (NaCl) and/or potassium chloride (KCl) and/or chloride lithium (LiCl) in the advantageous case where hydrochloric acid (HCl) is used in the step of precipitation of the terephthalic acid 310, or sodium sulphate (Na₂SO₄) in the case where sulfuric acid (H₂SO₄) is used), is submitted, after the step of neutralization 350, to a step of recovery 380 comprising a step of electrolysis 360 and a possible step of combination 370 (which according to the specific embodiment, may or may not be involved), from which the same mineral acid is obtained (for example, HCl or H₂SO₄) which is used in the step of precipitation of the terephthalic acid 310 of the method according to the invention; the mineral acid thus obtained may be advantageously used in a further step of precipitation of the terephthalic acid 310 performed subsequently.

Advantageously, one or more alkali metal hydroxides IMA such as, for example sodium hydroxide (NaOH) and/or potassium hydroxide (KOH) and/or lithium hydroxide (LiOH), are obtained from the step of recovery 380; advantageously, such one or more alkali metal hydroxides IMA (such as, for example sodium hydroxide (NaOH) and/or potassium hydroxide (KOH) and/or lithium hydroxide (LiOH)) may be at least partially reused in a depolymerization step 110 performed subsequently, preferably after being mixed with any reaction water AR and water-soluble organic reaction solvent SOIR during a possible mixing step 130, and/or may be at least partially employed in a step of neutralization 350 performed subsequently.

In an advantageous first embodiment, illustrated, for example in FIG. 1, wherein the step of precipitation of the terephthalic acid 310 uses the hydrochloric acid (HCl) as the mineral acid, the step of recovery 380 advantageously comprises a first step of electrolysis 361, wherein the neutral aqueous solution SAN, obtained from the neutralization of the acid aqueous solution SAA in the step of neutralization 350, is submitted to an electrolysis process preferably performed in a two-compartment cell. Chlorine (Cl₂) and hydrogen (H₂) in the gaseous state are obtained from this process, which are then reacted with each other in a first step of combination 371 advantageously provided in this step of recovery 380, at the output of which hydrochloric acid (HCL) is obtained.

In this first advantageous embodiment, also one or more alkali metal hydroxides IMA, such as for example sodium hydroxide (NaOH) and/or potassium hydroxide (KOH) and/or lithium hydroxide (LiOH), are also advantageously obtained from the first step of electrolysis 361, which can be advantageously reused, at least in part, in a step of neutralization 350 and/or a depolymerization step 110 and/or a possible mixing step 130, which are performed subsequently to said first step of electrolysis 361.

In a second advantageous embodiment, illustrated for example in FIG. 10, in which sulfuric acid (H₂SO₄) is used as the mineral acid in the step of precipitation of the terephthalic acid 310, the step of recovery 380 advantageously comprises a second step of electrolysis 362, in which the neutral aqueous solution SAN, obtained from the neutralization of the acid aqueous solution SAA in the step of neutralization 350, is submitted to an electrolysis process preferably performed in a three-compartment cell. Oxygen (O₂) and hydrogen (H₂) in the gaseous state, sulfuric acid (H₂SO₄) and one or more alkali bases, such as, for example one or more alkali metal hydroxides IMA, are obtained from this process such as, for example, sodium hydroxide (NaOH) and/or potassium hydroxide (KOH) and/or lithium hydroxide (LiOH); advantageously, oxygen (O₂) and hydrogen (H₂) in the gaseous state are then reacted with each other in a second step of combination 372, advantageously provided in the step of recovery 380, so as to obtain water.

Advantageously, the one or more alkali metal hydroxides IMA, e.g. sodium hydroxide (NaOH) and/or potassium hydroxide (KOH) and/or lithium hydroxide (LiOH), obtained in the second step of electrolysis 362 may advantageously be reused, at least in part, in a step of neutralization 350 and/or a depolymerization step 110 and/or a possible mixing step 130 performed subsequent to said second step of electrolysis 362.

Thus, it was found that the invention solves the task and purposes set forth above, in that the method according to the invention allows for the recycling of a textile waste comprising a cellulosic component, e.g. cotton fibres, and a polyester component, by effectively separating the same by chemical means.

The method according to the invention has the advantage of further comprising the removal of any contaminants from the cellulosic component and the polyester component, such as coloured pigments, thus returning the output of the substantially pure and easily reusable cellulosic component and the monomers of the polyester component.

In addition, the method according the invention lends itself to implementation on an industrial scale and is relatively simple and inexpensive.

Another advantage of the method according to the invention is sustainability; in fact, the auxiliary chemical substances and water employed in some steps of this method lend themselves to being recovered and reused in other steps of the same method performed later, thus minimizing the need for replenishment of these substances and the release of them into the environment.

Finally, it is clear that the method according to the invention described herein is susceptible to numerous modifications or variations, all of which are within the scope of the invention; moreover, all of the details are replaceable by technically equivalent elements without departing from the scope of the appended claims.

1) Method for the recycling of a textile waste (ST) comprising a cellulosic component and a polyester component, comprising the following steps:
a depolymerization step, comprising submitting said textile waste (ST) to a depolymerization reaction by basic hydrolysis of said polyester component by means of a solvolytic mixture (MS) which comprises an alkali metal hydroxide (IMA) and at least a water-soluble organic reaction solvent (SOIR), until obtaining a biphasic mixture (MB) comprising an initial solid phase (FSI), containing a terephthalic acid salt (SAT) and cellulosic fibres, and an initial liquid phase (FLI), containing said water-soluble organic reaction solvent (SOIR);
a first step of solid-liquid separation, comprising the removal of at least a part of said initial liquid phase (FLI) from said biphasic mixture (MB);
a step of treatment with organic solvent, wherein said initial solid phase (FSI), following said first step of solid-liquid separation, is treated with an organic washing solvent (SOL) until removing from said initial solid phase (FSI) one or more possible contaminants present therein, and obtaining a solid purified phase (FSP) containing said terephthalic acid salt (SAT) and said cellulosic fibres;
a second step of solid-liquid separation, wherein said solid purified phase (FSP) is separated from said organic washing solvent (SOL) and from said one or more possible contaminants dissolved therein;
a step of treatment with water, comprising bringing said solid purified phase (FSP) into contact with washing water (AL) until solubilizing said terephthalic acid salt (SAT) contained therein, and obtaining, by the removal of said terephthalic acid salt (SAT), a final solid phase (FSF) comprising said cellulosic fibres.

2) The Method, as in claim 1, wherein said organic washing solvent (SOL) is or comprises at least one of the following substances:
a glycol C₂-C₈,
an alcohol C₁-C₆ or a mixture of alcohols C₁-C₆,
a ketone C₃-C₈ or a mixture of ketones C₃-C₈,
an ether or a mixture of ethers,
an alkane C₅-C₈ or a mixture of alkanes C₅-C₈.

3) The method, as in claim 1, wherein said water-soluble organic reaction solvent (SOIR) and said organic washing solvent (SOL) are or comprise the same substance or the same mixture of substances.

4) The method, as in claim 1, wherein said step of treatment with organic solvent comprises one or more sub-steps of solid-solvent mixing, wherein said initial solid phase (FSI) is added and mixed with said organic washing solvent (SOL), each followed by a sub-step of solid-solvent separation, wherein said organic washing solvent (SOL) is removed, at least partially, from said initial solid phase (FSI).

5) The method, as in claim 4, wherein said one or more sub-steps of solid-solvent mixing comprise two or more sub-steps of solid-solvent mixing, and wherein the purity of the organic washing solvent (SOL), used in said two or more sub-steps of solid-solvent mixing, increases from the first to the last of said two or more sub-steps of solid-solvent mixing.

6) The method, as in claim 4, wherein said organic washing solvent (SOL) used in the last of said one or more sub-steps of solid-solvent mixing comprises a portion of fresh organic washing solvent (SOLF), and a portion of recovered organic washing solvent (SOLR) obtained removing it from a solid purified phase (FSP) in a second step of solid-liquid separation, performed before said last of said one or more sub-steps of solid-solvent mixing.

7) The method, as in claim 1, comprising, after said step of treatment with water, a step of water removal comprising removing from said final solid phase (FSF) said washing water (AL) if present therein.

8) The method, as in one or more of previous claim 7, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).

9) The method, as in claim 8, wherein said one or more sub-steps of solid-water mixing comprise two or more sub-steps of solid-water mixing, and wherein the purity of said washing water (AL) used in said two or more sub-steps of solid-water mixing increases from the first to the last of said two or more sub-steps of solid-water mixing.

10) The Method, as in claim 8, wherein, said washing water (AL) used in the last of said one or more sub-steps of solid-water mixing comprises a portion of fresh washing water (ALF) and a portion of recovered washing water (ALR) removed from a final solid phase (FSF) in a step of water removal of a method for the recycling of a textile waste (ST), performed before said last of said one or more sub-steps of solid-water mixing.

11) The method, as in claim 1, wherein, during said step of treatment with water, said washing water (AL) is at least partially removed from said purified solid phase (FSP), and wherein the washing water so removed (SA-SAT) is submitted to a step of precipitation of the terephthalic acid wherein it is reacted with a mineral acid, to form a suspension of initial terephthalic acid (ATI) in an acid aqueous solution (SAA), said method comprising a step of separation of the terephthalic acid, wherein said initial terephthalic acid (ATI) is removed from said acid aqueous solution (SAA), said acid aqueous solution (SAA) being then submitted to a step of neutralization of said method, wherein said acid aqueous solution (SAA) is brought into contact with an alkaline base to form a salt of an alkali metal and obtain a neutral aqueous solution (SAN), said method comprising submitting said neutral aqueous solution (SAN) to a step of recovery, comprising a step of electrolysis and an optional step of combination, from which the same mineral acid that is used in said step of precipitation of the terephthalic acid and the same alkaline base that is used in said step of neutralization are obtained, said mineral acid thus obtained being used in a further step of precipitation of the terephthalic acid, performed subsequently.

12) The method, as in claim 1, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).

13) The method, as in claim 2, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).

14) The method, as in claim 3, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).

15) The method, as in claim 4, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).

16) The method, as in claim 5, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).

17) The method, as in claim 6, wherein said step of treatment with water comprises one or more sub-steps of solid-water mixing, wherein said purified solid phase (FSP) is added and mixed with washing water (AL), each followed by a sub-step of solid-water separation, wherein said washing water (AL) is removed, at least partially, from said purified solid phase (FSP).
